(19) United States
(12) Patent Application Publication
(10) Pub. No.: US 2008/0255567 A1
(43) Pub. Date: Oct. 16, 2008

(54) Spinal distraction system

(76) Inventor: Joseph Accordino, Astoria, NY (US)

Correspondence Address: CASELLA & HESPOS, 274 MADISON AVENUE, NEW YORK, NY, 10016, US

(21) Appl. No.: 12/082,255

(22) Filed: Apr. 10, 2008

Related U.S. Application Data
(60) Provisional application No. 60/922,725, filed on Apr. 10, 2007.

(51) Int. Cl. A61B 17/58

(52) U.S. Cl. 606/90

(57) ABSTRACT

A spinal distraction apparatus includes substantially parallel fixed and movable legs, each of which has opposite front and rear ends. A rear rod projects rigidly from the rear end of the fixed leg and passes slidably through the rear end of the movable leg. A front rod extends rigidly from the fixed leg and is parallel to the rear rod. The front rod is formed with an array of external threads and nuts are threadedly engaged on the external threads for engaging opposite sides of the movable leg at locations adjacent the front rod. Thus, the position of the movable leg relative to the fixed leg can be controlled by adjusting the positions of the nuts. Front ends of the fixed and movable legs have attachments mounted thereto for engaging pedicle screws or other structures used in spinal distraction surgery.

This application claims priority on U.S. Provisional Application No. 60/922,725 filed on Apr. 10, 2007.

### BACKGROUND OF THE INVENTION

1. Field of the Invention

The invention relates to an apparatus for distracting portions of the spine during various types of spinal surgery.

2. Description of the Related Art

Injury, disease and various other conditions can lead to very painful and debilitating back problems. These problems often require surgical intervention so that the patient can resume a pain-free active life. Some spinal surgery fuses two or more vertebrae in a fixed relationship to one another. These surgical procedures can be carried out at different locations along the spine and can approach the spine from different angular orientations. The various types of spinal fusion include posterior lumbar interbody fusion, transforaminal lumbar interbody fusion, anterior lumbar interbody fusion and anterior cervical inter body fusion. Many of these procedures involve implanting prosthetic components into or near the spine such as screws and structural cages. Other spinal surgery is carried out to replace or supplement the natural disc between adjacent vertebrae. These procedures include lumbar total disc arthroplasty and cervical total disc arthroplasty. Still other spinal surgery includes single or multilevel corpectomy and surgical revisions to any of the above-identified described procedures.

The spinal surgery procedures described above require the surgeon to distract two or more vertebrae relative to one another. The distraction may be required to achieve a required spacing between adjacent vertebrae prior to fusing the vertebrae relative to one another. In other instances, the distraction may be carried out so that the surgeon can properly implant a replacement disc between adjacent vertebrae.

The prior art includes many different types of devices for performing spinal distraction. The particular type of spinal distraction device employed by a surgeon typically has varied in accordance with the type of surgery being performed and/or the type of prosthetic component (e.g. screw, cage, disc) being implanted. Many manufacturers of prosthetic components have dedicated spinal distraction apparatus for their particular prosthetic component. As a result, surgeons must be familiar with various different types of spinal distraction systems. Furthermore, a hospital must maintain inventories of many different types of spinal distraction apparatus and must assure that the particular spinal distraction apparatus that is made available to the surgeon is appropriate for the particular type of spinal surgery being performed and/or for the particular brand of prosthetic component.

Many of the available spinal distraction systems are very complicated to use. Improper use of a spinal distractor can have serious and permanent medical consequences. Furthermore, many prior art surgical distraction devices are large and adversely limit access of the surgeon to the desired intervertebral space.

In view of the above, it is an object of the invention to provide a spinal distraction system that can be used for many different types of spinal surgery and with many different types of prosthetic components.

It is another object of the subject invention to provide a spinal distraction system that is accurate and uncomplicated.

A further object of the subject invention is to provide a spinal distraction system that is easy to use.

Still another object of the subject invention is to provide a spinal distraction system that is adaptable for all approaches to the spine.

It is a further object of the invention is to provide a spinal distraction system capable of spanning several levels of the spine when require for multi-level procedure.

Another object of the subject invention is to provide a spinal distraction system that is small and that does not limit surgical access to the desired intervertebral space.

### SUMMARY OF THE INVENTION

The invention relates to a spinal distraction system that has first and second distractor legs. The first distractor leg preferably is stationary while the second distractor leg preferably is movable. Each distractor leg has a front end and a rear end. The front end is configured to receive one of a plurality of attachments for engaging and cooperating with a pedicle screw or other such prosthetic component that is being used in the surgical procedure. At least one hinge preferably is provided between the front and rear ends of each of the distractor legs. Each hinge is configured to pivot about an axis that extends substantially parallel to the axis of the spine. In a preferred embodiment, each leg has two hinges disposed to rotate about parallel axes.

The spinal distraction apparatus further includes first and second substantially parallel rods that extend transverse to the legs. The rods preferably are mounted to the first or fixed leg and extend through and beyond the second or movable leg. Ends of the rods remote from the first leg preferably are connected rigidly to one another to ensure parallelism between the rods when the rods are subjected to forces during spinal distraction. The first rod preferably is disposed substantially at the rear end of each leg and preferably has smooth exterior. The second rod preferably is between the first rod and the hinges and is formed with an array of external threads.

The movable leg preferably has first and second apertures extending therethrough in directions transverse to the longitudinal direction of the movable leg. The first and second apertures are disposed to permit the movable leg to telescope over the first and second rods. The first aperture in the movable leg preferably is in close sliding engagement with the smooth exterior surface of the first rod. The first aperture in the movable leg may be lined with a low friction material to ensure smooth sliding engagement between the first aperture and the first rod. For example, the first aperture may have a nylon or Teflon lining. The internal cross-sectional dimensions of the first aperture and the external cross-sectional dimensions of the first rod are selected to minimize tilting or binding of the movable leg on the first rod and to maintain substantial parallelism between the fixed and movable legs. The second aperture through the movable leg need not closely engage the threaded exterior surface of the second rod.

First and second nuts preferably are threadedly engaged on the second rod. The first nut is disposed between the fixed and movable legs. The second nut is disposed between the second leg and the connection between the ends of the rods. The nuts can be rotated on the second rod to fix the position of the movable leg relative to the fixed leg and/or to limit the range of movement of the fixed leg relative to the movable leg. For example, the nuts can be disposed so that movement of the movable leg in one direction is prevented but limited movement of the movable leg in the opposed direction is permitted. Washers may be disposed on the second rod between the movable leg and the nuts.

The front end of each leg is configured to receive an attachment. The attachments preferably extend at an angle to the longitudinal direction of the legs. The attachments can be extensions that enable the overall length of the legs to be extended. The extensions are selected in view of the size or other physical characteristic of the patient and in view of the type of surgery that is being performed. For example, an extension for an anterior lumbar interbody fusion may be longer than an extension for a posterior lumbar interbody fusion.

The spinal distraction apparatus further includes a plurality of distraction attachments. Each distraction attachment includes a mounting end and a working end. The mounting ends of the distraction attachments are mountable to either the front end of one of the legs or to the front end of one of the extensions. The mounting ends of all of the attachments preferably are substantially identical to one another so that the distraction attachments can be selected and interchanged in accordance with the surgical procedure. The working end of each distraction attachment is configured in accordance with the size, shape and characteristics of the pedical screw, rod or other prosthetic component and in accordance with the particular types of instrumentation that will be used for the surgery.

The spinal distraction apparatus is employed by determining the longitudinal length required for the rods and the longitudinal-lengths required for the legs. Short rods are appropriate for physically small patients and for instances where the surgeon will be working only on vertebrae that are adjacent to one another or near one another. Longer rods will be required for larger patients or for surgical procedures being carried out at vertebrae that are spaced considerably apart. The length required for the legs is dependent at least partly upon the size of the patient and the surgical approach. The leg extensions can be selected and attached to the front ends of the respective legs if the surgeon determines that additional leg length is necessary. The leg extensions preferably are configured for a quick connect (e.g. snap-fit attachment).

The spinal distraction apparatus is configured by attaching the appropriate distraction attachment to the front end of the respective leg or to the front end of the extension. The surgeon then may adjust the position of the movable leg along the rods and relative to the fixed leg. The nuts then are rotated on the second rod to position the movable leg approximately. The working end of the respective distraction attachments then are engaged with the appropriate pedicle screws, rods or the like. The movable leg then may be urged farther away from the fixed leg to distract the appropriate vertebrae amounts deemed necessary by the surgeon.

The spinal distraction apparatus may be used with a plier-like rod holder for securely gripping a rod used in the distraction or surgical procedure. The distraction device may further include a plier-like sliding leg distractor. The sliding leg plier-like distractor can be positioned near the smooth outer surface of the first rod and can be squeeze or otherwise actuated for exerting forces on the legs for urging the legs farther apart.

### BRIEF DESCRIPTION OF THE DRAWINGS

FIG. 1 is a perspective view of the spinal distraction apparatus of the subject invention.

FIG. 2 is a side elevational view of the spinal distraction apparatus shown in FIG. 1.

FIG. 3 is a front elevational view of the spinal distraction apparatus with the attachments removed therefrom.

FIG. 4 is a front elevational view of a pair of attachments for use with the apparatus shown in FIG. 3.

FIG. 5. is a rear elevational view of the apparatus shown in FIG. 3, with the attachments removed.

FIG. 6 is a rear elevational view of the pair of attachments shown in FIG. 4.

FIG. 7 is a side elevational view showing the final distraction apparatus use with pedicle screws mounted in a spine.

FIG. 8 is a rear elevational view of the apparatus use with the pedicle screws mounted in the spine as shown in FIG. 7.

FIG. 9 is a side elevational view of a portion of the spinal distraction apparatus with a distraction attachment in the form of a rod attachment for a top loading pedicle screw.

FIG. 10 is a front elevational view of the attachment shown in FIG. 9.

FIGS. 11 and 12 are side and front elevational views showing a polyaxial ring attachment.

FIG. 13 is a side elevational view of a pin.

FIG. 14 is a side elevational view of spinal distraction apparatus used with an anterior cervical distraction attachment.

FIG. 15 is a front elevational view of the anterior cervical distraction attachment shown in FIG. 14.

FIG. 16 is a top plan view of the anterior cervical distraction attachment of FIGS. 14 and 15.

FIG. 17 is an exploded elevational view of a cervical pin for use with a cervical distraction attachment.

FIG. 18 is a front elevational view of an extension for use with the spinal distraction apparatus.

FIG. 19 is a side elevational view of the extension shown in FIG. 18.

### DETAILED DESCRIPTION OF THE PREFERRED EMBODIMENT

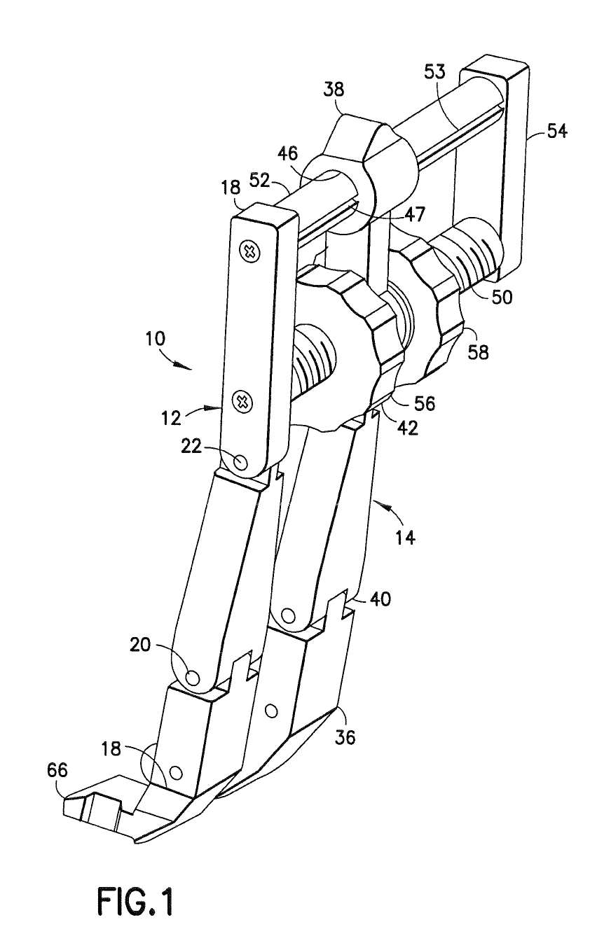

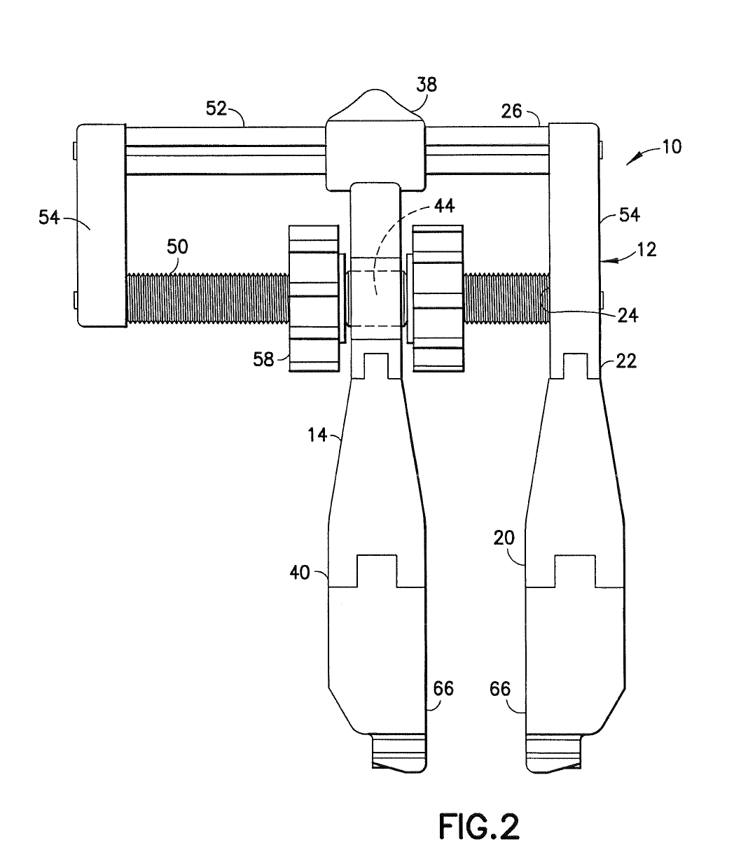

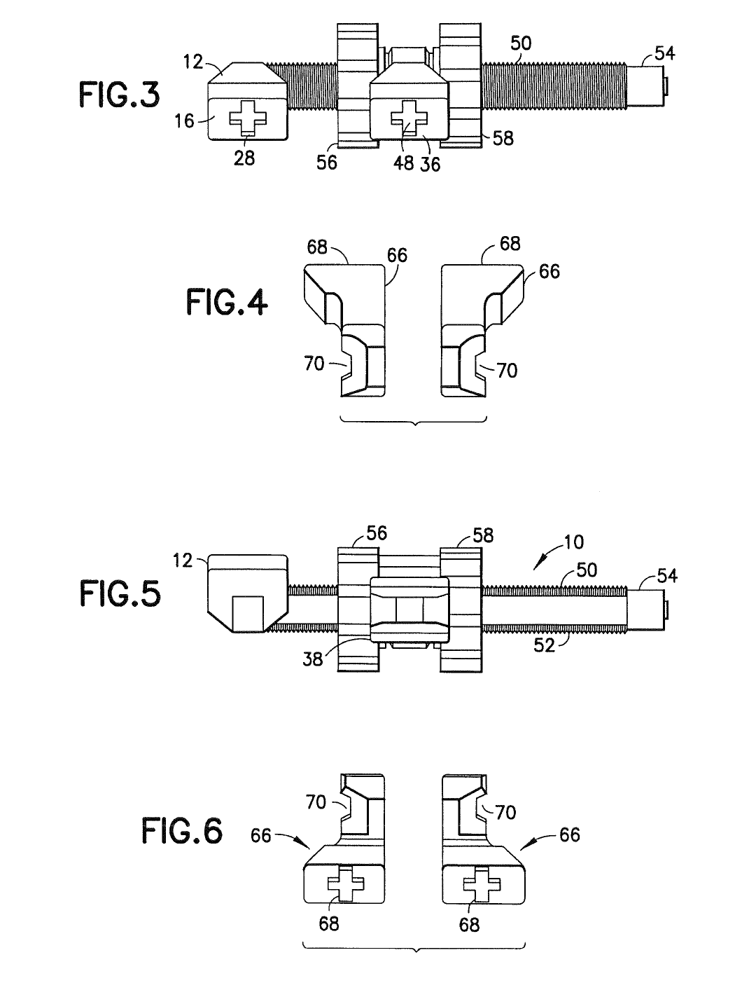

A spinal distraction apparatus in accordance with one embodiment of the subject invention is identified generally by the numeral 10 in FIGS. 1-6. The spinal distraction apparatus 10 includes a fixed leg 12 and a movable leg 14 that are aligned substantially parallel to one another. The fixed leg 12 has opposite front and rear ends 16 and 18. A front hinge 20 is disposed between the front and rear ends 16 and 18 of the fixed leg 12 and a rear hinge 22 is disposed between the rear end 18 of the fixed leg 12 and the front hinge 22. The front and rear hinges 20 and 22 are configured to rotate about parallel axes that extend in left-to-right directions in FIG. 2. A front rod socket 24 is formed in one side of the fixed leg 12 between the rear end 18 and the rear hinge 22. A rear rod socket 26 is formed in the fixed leg 12 substantially adjacent the rear end 18 and on the same side of the fixed leg 12 as the front rod socket 24. An attachment socket 28 is formed in the front end 16 of the fixed leg 12.

The movable leg 14 is similar to the fixed leg 12. More particularly, the movable leg 14 has opposite front and rear ends 36 and 38. Front and rear hinges 40 and 42 are formed between the front and rear ends 36 and 38 and are configured to rotate about axes that are coaxial with the rotational axes defined by the hinges 20 and 22. A front rod aperture 44 extends entirely through the movable leg 14 at a location between the rear end 38 and the rear hinge 42. A rear rod aperture 46 extends entirely through the movable leg 14 at a position near the rear end 38. The front and rear rod apertures 44 and 46 are illustrated most clearly in FIG. 1. The rear rod aperture 46 is smoothly cylindrical, but includes two diametrically opposite ribs 47 extending parallel to the axis of the cylinder. The rear rob aperture 46 preferably is lined with a material that has low coefficient of sliding friction, such as nylon or Teflon. Portions of the movable leg 14 that have the rear rod aperture 46 are significantly wider than portions near the front rod aperture 44. More particularly, the cylindrical rear rod aperture 46 has a significantly greater axial dimension than the front rod aperture 44. An attachment socket 48 is formed in the movable leg 14 adjacent to the front end 36 of the movable leg 14.

The spinal distraction apparatus 10 further includes front and rear rods 50 and 52. The front rod 50 projects non-rotatably from the front rod socket 24 of the fixed leg 12 and is substantially perpendicular to the longitudinal direction of the fixed leg 12. The exterior of the front rod 50 includes an array of external threads. Portions of the front rod 50 spaced from the front rod socket 24 pass through the front rod aperture 44 in the movable leg 14. The rear rod 52 projects non-rotatably from the rear rod socket 26 in the fixed leg 12. The rear rod 52 is substantially parallel to the front rod 50 and is characterized by a smooth cylindrical exterior surface with two diametrically opposite grooves dimension for receiving the ribs 47 of the rear rod aperture 46. Portions of the rear rod 52 that project from the rear rod socket 26 are in close sliding engagement with rear rod aperture 46 that passes through the movable leg 14.

A connector 54 extends between and securely connects ends of the front and rear rods 50 and 52 opposite the fixed leg 12. With this arrangement, the movable leg 14 can be slid along the front and rear rods 50 and 52 towards and away from the fixed leg 12. The close sliding engagement of the rear rod 52 with the rear rod aperture 46 assures substantial parallelism between the fixed and movable legs 12 and 14. A distracting nut 56 is threadly mounted on the front rod 50 between the fixed leg 12 and the movable leg 14. A locking nut 58 is threadly mounted on the front rod 50 between the movable leg 14 and the connector 54. Threaded movement of the distracting nut 56 away from the fixed leg 12 can urge the movable leg 14 away from fixed leg 12 to perform the final distraction or can hold the movable leg 14 in a distracting position after the movable leg has been positioned by other means. The locking nut 58 can be threaded to an appropriate position for preventing movement of the movable leg 14 away from the fixed leg 12 or for permitting a controlled movement of the movable leg 14 away from the fixed leg 12. Washers preferably are mounted on the front rod 50 between the movable leg 14 and nuts 56 and 58.

The front and rear rods 50 and 52 are removably mountable in the front and rear sockets 24 and 26 respectively and are removably connected to the connector 54. The front and rear rods 50 and 52 shown in FIG. 1 can be replaced by longer or shorter rods in accordance with the size and other physical characteristics of the patient and in accordance with the nature of the surgery being performed. For example, surgery that transcends several spaced-apart vertebrae will require longer rods 50 and 52.

The spinal distraction attachment apparatus 10 further includes extension attachments 60, as shown in FIGS. 18 and 18. The extension attachments 60 can be mounted to the attachment sockets 28, 48 in the front ends 16, 36 of the fixed or movable legs 12 or 14. The extension attachments 60 each have a mounting end 62 configured for secure mounting in the socket 28 or 48 and a socket end 64 configured for engaging a distraction attachment as described herein. The extension attachments 60 have different optional lengths between their opposite ends 62 and 64. An extension attachment 60 of appropriate length is selected by the surgeon in accordance with the nature of the surgical procedure that is being performed and the size of the patient.

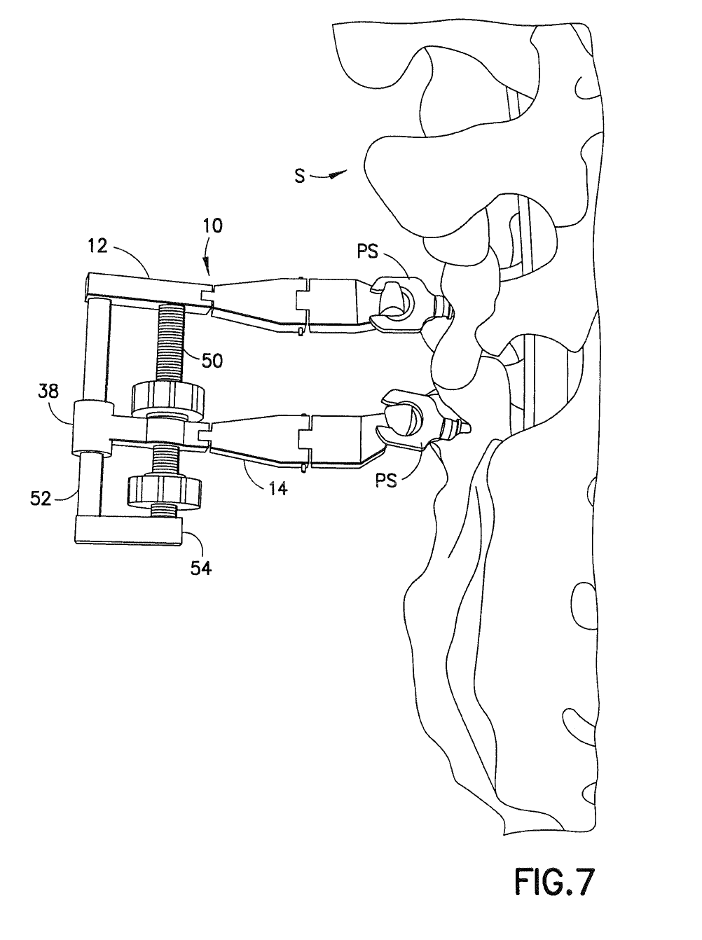

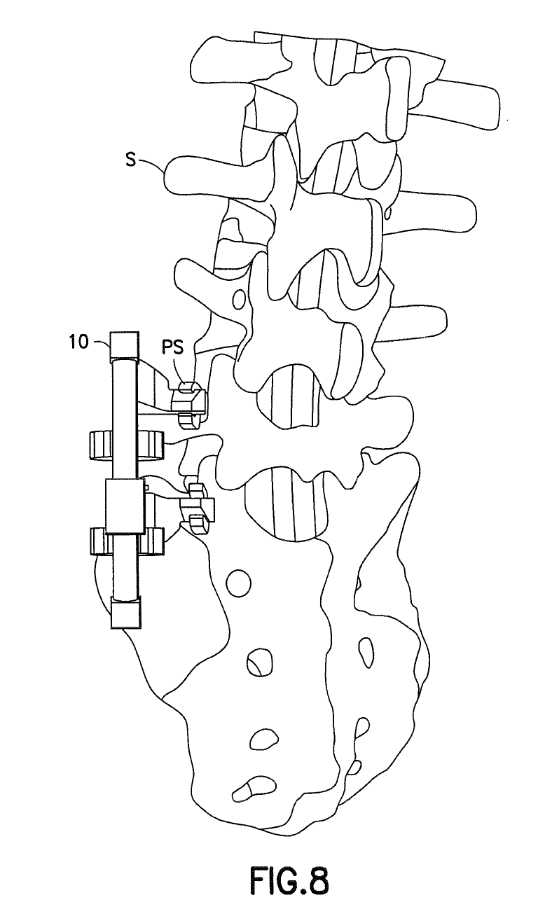

The spinal distraction apparatus 10 further includes distraction attachments that can be mounted to the front end 16 or 36 of the fixed or movable leg 12 or 14 or to the socket 64 at the end of the extension attachment 60. The distraction attachments have different configurations in accordance with the type of surgery being performed, the characteristic of the instrumentation that is being used and the shapes of the pedicle screws that are being used. For example, FIGS. 1-8 show a pedicle screw distraction attachment 66. The pedicle screw distraction attachment 66 has a mounting end 68 as shown in FIG. 6 and configured for mounting in the socket 28, 48 at the front end 16 or 36 of the fixed or movable legs 12 or 14, or in the socket 64 in any of the extension 60. The pedicle screw distraction attachment 66 also includes a working end 70 as shown in FIG. 4. The working end 70 of the pedicle screw distraction attachment 66 is aligned at a right angle to the front to rear direction of the respective legs 12 or 14 and includes a 60 degree lateral to medial reach section between the ends. The hinges 20, 22, 40, 42 in the fixed and movable legs 12 and 14 allow for medial/lateral movement of the pedicle screw distraction attachments 66. Medial movement achieved by the hinges 20, 22, 40, 42 will position the assembly of the legs 12, 14 and the rods 50, 52 contralateral to the point entry to the disc space, as shown in FIGS. 7 and 8 with the legs 12 and 14 extending just over the head of the pedicle screw PS mounted in the spine S. Lateral movement achieved by the hinges 20, 22, 40, 42 will position the frame defined by the legs 12, 14 and rods 50, 52 outside of the incision, as shown in FIG. 8, with the addition of the extension 60 if required by the anatomy of the patient. The working end 70 of the pedicle screw distraction attachment 66 has a rectangular bevel to an extremely low profile with a partial hexagonal contact region that preferably is lined with nylon, Teflon or the like. The bevels provide a surface that will lean towards an upright position when distraction force is applied. The nylon, Teflon or the like will allow for a steady grip on the pedicle screw PS, as shown in FIGS. 7 and 8, without damaging the pedicle screw PS. This profile allows for placement of the working end 70 under the head of the pedicle screw PS without impinging upon the bone beneath the head of the pedicle screw. The pedicle screw distraction attachments 66 shown in the figures are usable with all types of pedicle screws for posterior lumbar interbody fusion and transforaminal lumbar interbody fusion.

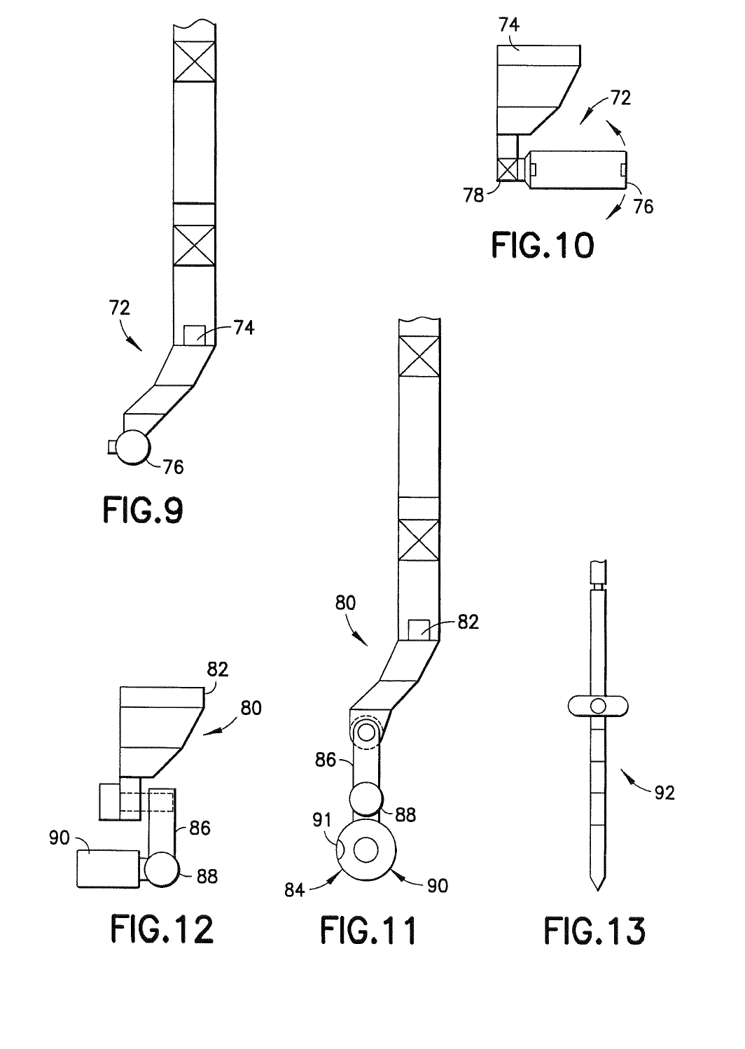

FIGS. 9 and 10 show rod attachments 72. The rod attachment 72 has a mounting end 74 identical to the mounting ends 68 of the pedicle screw distraction attachments 66 and on the extensions 60. The rod attachments 72 further include a working end 76. The rod attachment 72 extends initially at an approximately 60 degree angle from the longitudinal direction of the leg 12, 14 and then extends further to an approximately 45 degree reach. The rod attachment 72 includes a hinge 78 that permits cephalad/caudal movement as the attachment 74 is placed in the head of a screw. This attachment 72 can be used as an adjustable distraction rod as the desired interverbral space is determined. The space is maintained by adjusting the position of the distraction nut 56 against the movable leg 14. This sliding capability of the movable leg allows for movement of the vertebral bodies as work continues in the disc space, thereby eliminating end plate destruction and facilitating structural graft placement. The attachment 72 described and illustrated herein can be provided with working ends 76 that are of various cross-sectional dimensions according to the screw system that is being used. Tabs preferably are provided at both ends and function as a stop for the head of the screw. The locking mechanism for the screw will be applied in a provisional manner to close the distraction rod in place. The rod attachments 72 can be used with polyaxial and monoaxial pedicle screws that receive the rod directly to the head (top loading) for posterior lumbar interbody fusion and transforaminal lumbar interbody fusion.

A polyaxial ring attachment is illustrated in FIGS. 11 and 12 and is identified generally by the numeral 80. The polyaxial ring attachment 80 is used for lumbar total disc arthroplasty and anterior lumbar interbody fusion and is used in cooperation with smooth pins placed in the vertebral bodies. The polyaxial ring attachment has a mounting end 82 identical to the mounting ends of the other attachments and has a working end 84. Portions of the attachment 80 between the ends initially define a 60 degree reach that then extends to a 45 degree reach, in much the same way as the rod attachments 72 described above and illustrated in FIGS. 9 and 10. A partially threaded machine screw is inserted through the working end 84 at a 90 degree angle. The machine screw is threaded into the ring attachment rod 86 and provides for medial/lateral motion of the ring attachment rod. The ring attachment rod 86 has a ball and socket joint 88 with the socket at the end of the ring attachment rod 86 and with the ball extending from the ring 90. Thus, the ring 90 can be disposed in any desired plane. The polyaxial ring attachment 80 is used with smooth pins such as the stieman pin 92 shown in FIG. 13. The pin 92 has a pointed end and an oval smooth stop set at a level determined by the anatomy of a patient. The stop keeps the distraction rings from infringing upon vascular structures and allows the pin to be driven to the center of the vertebral bodies. The stop has a set screw at a 90 degree angle to the pin. The pins 92 are inserted slightly oblique in the axial plane and slightly convergent in the sagittal plane to the center of the vertebral body superior and inferior to the space of focus. The ring 90 has a set screw 91 inserted at a 70 degree angle from the proximal outer edge of the ring 90 to the inner opening thereof. The set screw 91 has a round distal end to lock the ring 90 in place on the pin 92. A universal motion tip screw driver may be use to tighten and losing the set screw after the ring 90 is placed on the pin 92 down to the oval stop. Extensions varying in length determined by the anatomy of the patient are added to place the spinal distraction apparatus 10 outside the area of focus. Longer rods 50 and 52 can be used for multilevel procedures including multilevel segmental fusion and single and multilevel corpectomy. The distraction rings 90 are attached to the pins 92 after the pins 92 have been placed in the vertebral bodies and the set screws 91 are tightened. The distraction now will open the anterior portion of the space of focus. Using slight compression at the proximal ends of the pins 92, the posterior portions of the two opposing vertebral bodies will open while the rings maintain the desire anterior opening. This arrangement is beneficial for optimum placement of intervertebral devices, with special consideration given to devices that have fixation teeth designed for placement at or near the posterior cortical ring of the vertebral bodies. This type of distraction is advantageous during the extraction of these devices as well. The polyaxial ring attachment 80 also can be used posterior with pedicle screws that have a post and connector to medialize the rod for posterior lumbar interbody fusion and transforaminal lumbar interbody fusion. The ring 90 can be attached to the post of the side loading pedicle screw with a locking nut tightened in a provisional manner to maintain contact of the distraction rings with the pedicle screw.

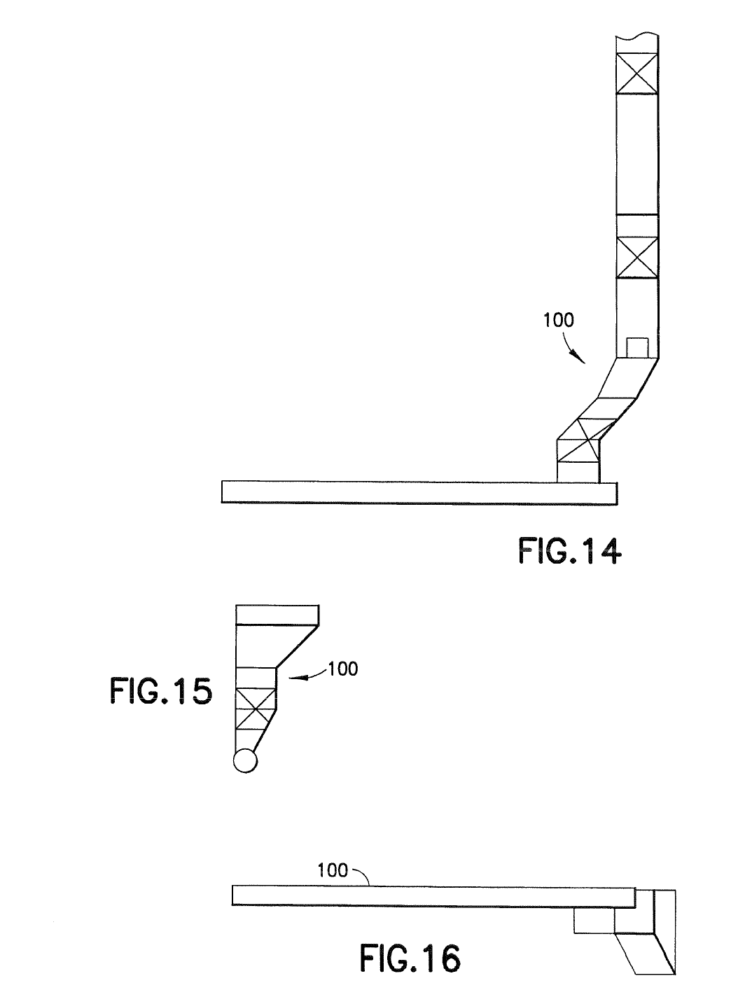

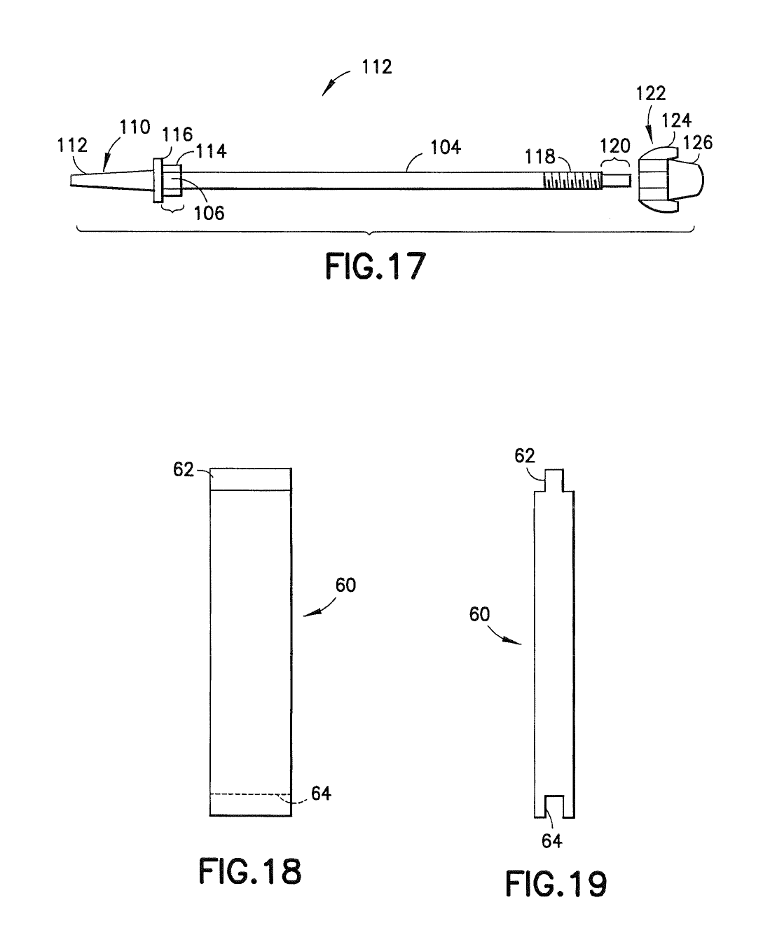

FIGS. 14-16 illustrate an attachment 100 for use during cervical total disc arthroplasty and anterior cervical interbody fusion procedures. The attachment 100 of FIGS. 14-16 is used with a distraction pin assembly 102 as shown in FIG. 17. The distraction pins 102 can be placed in the vertebral body at locations superior and inferior to the space of focus. The pin assembly 102 has a pin body 104 with a proximal end 108 and a distal end 106. A fitting 110 is mounted to the distal end 106 of the pin body 104. The fitting 110 has tapered self-tapping coarse threads 112 extending from the extreme distal end of the fitting 110 and a hex nut 114. A circular planar stop 116 is disposed proximally of the threads 112 and distally of the nut 114. The pin body 104 has a smooth outer surface extending a major part of the length from the distal end 106 towards the proximal end 108. However, an array of fine threads 118 is formed on the pin body 104 and a reduced diameter smooth tip 120 extends from the array of threads 118 to the proximal end 108. The short smooth tip 120 at the proximal end 108 facilitates placement of a locking nut 122 during threaded engagement of the locking nut 122 with the pin body 104. Hence, the locking nut 112 is not likely to be separated inadvertently from the pin body 104 even though the locking nut 122 is not engaged fully with the threads 118 during assembly and disassembly of the locking nut 112 to the pin body 104. The locking nut 122 has small wings 124 and dome 126 to facilitate assembly and disassembly. The locking nut 112 prevents sliding of the distraction canular on the pin assembly 102 while maintaining distraction. The pin assemblies 102 may be positioned using a canulated self-retaining driver at a diversion angle to provide sufficient lordosis when inserted into the parallel cannulas of the distractor. The frame defined by the arms 12, 14 and rods 50, 52 will extend away from the area of focus at an angle of approximately 90 degrees from the proximal ends 108 of the pin assemblies 102. This orientation permits an unobstructed visualization of the areas of focus and enables use of the microscope. The attachment also positions the frame to prevent interference with wound retraction. The combination of the anterior cervical distraction attachment 100 and the frame shown FIG. 1 is useful for multi-level cervical procedures, including corpectomy, with the addition of longer rods 50, 52.

What is claimed is:

1. A spinal distraction apparatus comprising:
a fixed leg having opposite front and rear ends;
a rear rod extending rigidly from the rear end of the fixed leg and aligned angularly to the fixed leg;
a front rod extending rigidly from a portion of the fixed leg between the rear rod and the front end of the fixed leg, the front rod being substantially parallel to the rear rod;
a movable leg having opposite front and rear ends and aligned substantially parallel to the fixed leg, the rear end of the movable leg being slidably engaged with the rear rod for permitting slidable movement of the movable leg towards and away from the fixed leg;
movement control members mounted movably on the front rod for selectively controlling a distance between the fixed and movable rods; and
attachments secured to the front ends of the respective fixed and movable legs or engaging structure elements mountable to a human spine for distracting vertebrae.

2. The apparatus of claim 1, further comprising a connector rigidly joining ends of the front and rear rods remote from the fixed leg.

3. The apparatus of claim 2, wherein the fixed and movable legs each include at least one hinge defining hinge axes substantially parallel to the front and rear rods.

4. The apparatus of claim 3, wherein the at least one hinge comprises rear hinges on the fixed and movable legs respectively with hinge axes aligned collinear with one other and front hinges on the fixed and movable legs with hinge axes thereof aligned substantially collinearly with one another, the front hinges being disposed between the front ends of the fixed and movable legs and the front rod, the rear hinges being disposed between the front hinges and the front rod.

5. The apparatus of claim 1, wherein the front rod includes an array of external threads, the movement control members including internally threaded nuts threadedly engaged on the front rod for movement along the front rod towards and away from the fixed leg.

6. The apparatus of claim 5, wherein the movable leg includes a front rod aperture, the front rod passing through the front rod aperture without engaging the front rod.

7. The apparatus of claim 1, wherein the front and rear rods are aligned substantially perpendicular to the fixed leg.

8. The apparatus of claim 9, wherein the rear rod and the rear end of the movable leg are formed with interengageable ribs and grooves for guiding movements of the movable leg along the rear rod.

9. The apparatus of claim 1, wherein the attachments are removably attached to the fixed and movable legs, each of set attachments having an end remote from the respective fixed and movable legs configured for engaging a pedicle screw.

10. The apparatus of clam 1, further comprising rod extensions removably attachable to ends of the front and rear rods remote from the fixed leg.

11. The apparatus of claim 1, wherein the attachments are elongate rods.

12. The apparatus of claim 1, wherein the attachments are polyaxial rings.

13. The apparatus of claim 1, wherein the attachment comprises at least one distraction pin assembly.
